(19) United States
(12) Patent Application Publication
(10) Pub. No.: US 2003/0165614 A1
(43) Pub. Date: Sep. 4, 2003

(54) Coating a medical implant using a pan coater

(76) Inventors: Henrik Hansen, Croshoa (IE); John Clarke, Kiniska (IE)

Correspondence Address: KENYON & KENYON, 1500 K STREET, N.W., SUITE 700, WASHINGTON, DC, 20005, US

(21) Appl. No.: 10/087,014

(22) Filed: Mar. 1, 2002

(51) Int. Cl. A61L 2/00; B05D 3/12; B05D 1/02

(52) U.S. Cl. 427/2.24; 427/242; 427/421

(57) ABSTRACT

Method and system for coating medical implants utilizing a pan coater to apply a coating is provided. A system for coating the implant may include a rotatable drum, a source or multiple sources of therapeutic coating, a spray nozzle for spraying the coating into the interior of the rotatable drum, a source of compressible fluid for drying the coating, a reservoir for recovery of excess therapeutic coating, and a processor. The processor in this embodiment may control the rotation of the rotatable drum, the time and volume of spray through the spray nozzle, and the time, volume and temperature of the flow of compressible fluid into the drum.

### FIELD OF THE INVENTION

The present invention generally regards the coating of workpieces. More particularly the present invention regards coating a medical implant using a pan coater.

### BACKGROUND

The positioning and deployment of medical implants within the body of a patient is a customary procedure of contemporary medicine. Medical implants may be used for numerous medicinal purposes including the reinforcement of recently re-enlarged lumens, the replacement of ruptured vessels, the reinforcement of weakened joints, and the delivery of therapeutic.

Coatings are often applied to medical implants to increase their effectiveness. These coatings may reduce the trauma suffered during the procedure, facilitate the implantation of the medical implant at the target site, and improve the post-procedure effectiveness of the implant. Expandable stents, stent grafts, balloon delivery systems, and aneurism coils are examples of medical implants that may be coated.

Expandable stents are tube-like medical implants that often have a mesh-like appearance and may be designed to support the inner walls of a lumen within the body of a patient. These stents are often positioned within a lumen and then expanded, sometimes under their own internal forces and other times through external forces placed upon them. Because of the direct contact of the stents with the inner walls of the lumen, stents, like other implants, have often been coated with various compounds and therapeutics to enhance their effectiveness. When these coatings are haphazardly applied or have somehow been removed during manufacture or subsequent handling the stents' effectiveness can be compromised. In fact, in certain circumstances, faulty stents can require a second unwanted procedure to remove and replace them.

Coating methods such as dip-coating or spray-coating have been used to coat stents and other medical implants. These methods are, however, difficult to control and often result in significant waste. Dip-coating can result in non-uniform application of the coating to the stents, making it difficult to predict the dosage of therapeutic that will be delivered when the stents are implanted at the target site. Spray-coating may be cost prohibitive due to the waste associated with the technique and the extremely high cost of certain therapeutics.

FIGS. 1 and 2 illustrate a coated stent before and after its expansion. FIG. 1 shows stent 11 in a closed, pre-deployment state. Here, the stent 11 has been previously dipped in a vat of therapeutic in the direction of arrow 16. In other words, the right side of the stent was the leading edge of the stent entering the dipping vat. As can be seen, the coating of stent 11 is heavier on the right side of the stent 11 than on the left side and covers each of the junctions 13 throughout the entire stent 11. As can also be seen, the coating becomes progressively thicker and covers more of the space between each of the struts 12 moving from the left side of the stent 11 to the right side of the stent 11. This increasing coating thickness is indicative of a stent 11 that has been dipped and let stand on one of its ends as the coating dries and adheres to it.

FIG. 2 shows the unevenly coated stent 11 of FIG. 1 in an expanded state as it may be after it is positioned within the body. FIG. 2 illustrates how the expansion of stent 11 has led to the cracking and crumbling of the unevenly applied coating 15. FIG. 2 also illustrates that the unevenly applied coating 15 has been removed from most if not all of the junctions 13 of the struts 12 after the stent has been expanded.

### SUMMARY OF THE INVENTION

The present invention regards coating a medical implant using a pan coater. A method in accord with one embodiment includes providing a rotatable drum and a spray nozzle in fluid communication with the rotatable drum. The method also includes placing one or more medical implants in the rotatable drum and rotating the drum to tumble the medical implant(s) while spraying a liquid material into the drum to coat the medical implant(s). The method may also include injecting an inert gas into the drum to dry the coating onto the medical implant(s), heating the drum and/or the inert gas to promote the drying process, and spraying additional coats of different materials onto the medical implant(s). The final steps of the process may include stopping the rotating drum and removing the now coated medical implant(s) from the drum.

Another embodiment of the present invention may include a computer readable medium storing instructions for operating a pan coater for coating medical implant(s). The instructions for the pan coater may include directions to rotate a drum containing the medical implant(s) and to spray a therapeutic (or therapeutics) into the drum through a spray nozzle while the drum is rotating. These instructions may also include directions to inject an inert gas into the drum to dry the coated medical implant(s) and to heat the drum and/or the inert gas to aid in the coating process.

### BRIEF DESCRIPTION OF THE DRAWINGS

FIG. 1 is an enlarged side view of a stent that has been unevenly coated with a coating.

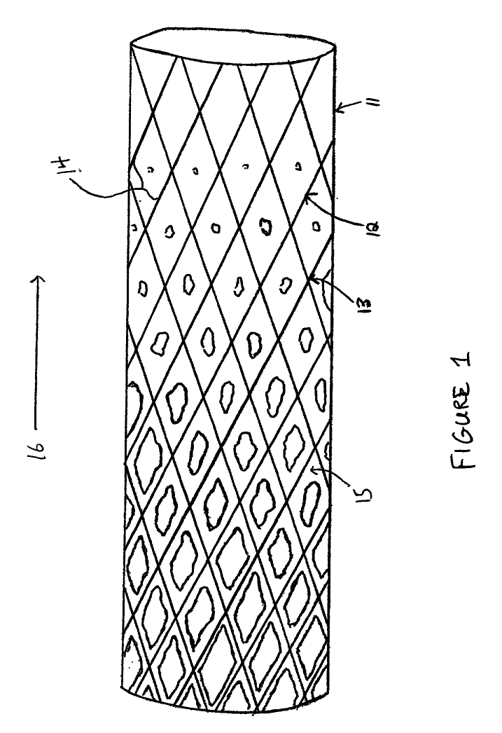

FIG. 2 is an enlarged side view of the stent of FIG. 1 in an expanded state, the uneven coating being broken and cracked at the junctions of the stent's struts.

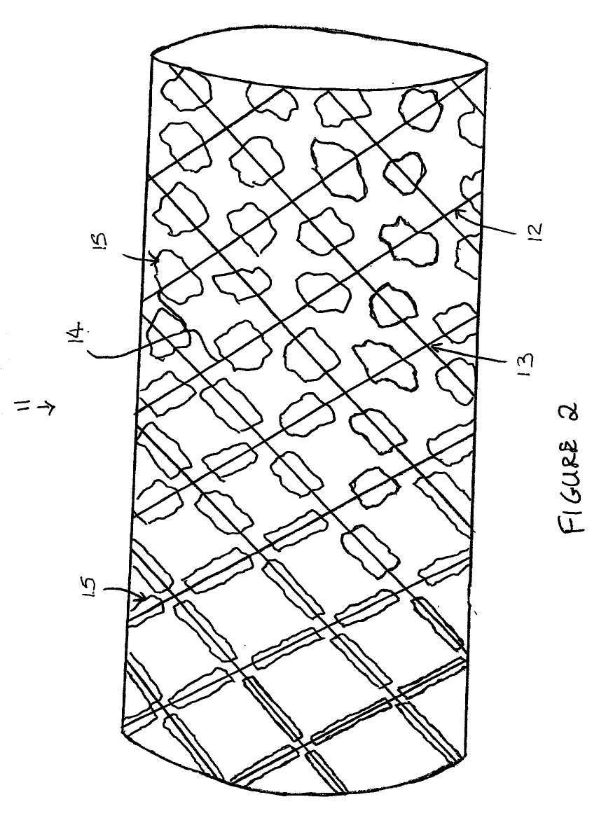

FIG. 3 is a schematic view of a pan coater in fluid communication with two coating sources in accord with one embodiment of the present invention.

FIG. 4 is a schematic view of a pan coater with an air suspension system in accord with another embodiment of the present invention.

FIG. 5 is a schematic view of a pan coater in accord with another embodiment of the present invention.

FIG. 6 is a schematic view of a pan coater in accord with another embodiment of the present invention.

FIG. 7 is a schematic view of a pan coater in accord with another embodiment of the present invention.

### DETAILED DESCRIPTION

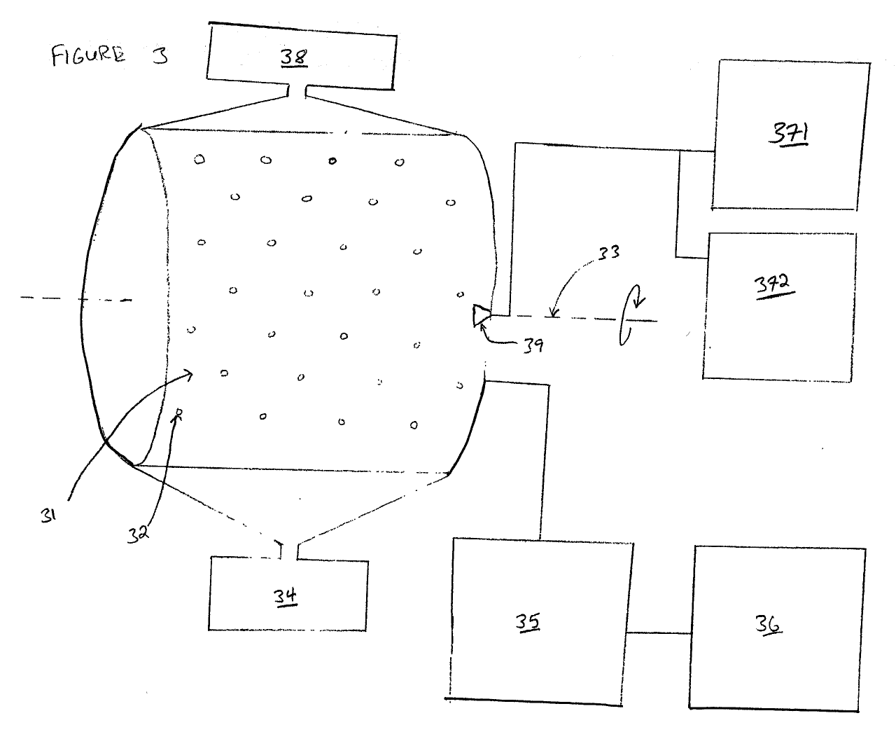

FIG. 3 illustrates a system for coating a medical implant using a pan coater in accord with one embodiment of the present invention. In this system, a rotatable drum 31 contains at least one medical implant (not shown) to be coated. These medical implants may be stents, catheters, patches, coils, prostheses and other types of implantable devices. The rotatable drum may be mounted such that it rotates about axis 33 and may have perforations 32 that may be used during the various coating and drying steps described below. The perforations 32 may extend completely through the drum 36 and may also be offset, having one set of openings on the outside of the drum 31 and a second set of openings on the inside of the drum 31, the second set offset but in fluid communication with the first set. The shape of the drum may be altered or extra elements included with it or attached to it to maximize coating efficiency and to prevent damage of the devices to be coated, e.g. baffles may be included on the inside of the drum or the drum may have a stellate cross-section.

The rotatable drum 31 may be rotated about axis 33 to ensure that all sides of the medical implant resident within the drum are exposed to therapeutic being sprayed from the spray nozzle 39. Alternatively, therapeutic may be forced through the perforations 32 into the drum 31 thereby creating a standing vat of therapeutic that the medical implant may tumble within in order to coat the medical implant in the drum.

The rotatable drum 31 may be controlled by or at least receive signals from a processor 35. The processor 35, which may contain internal non-volatile storage media for storing its instructions, may send control signals to the spray nozzle 39 and to any other component or device necessary for coating an implant placed into the drum 31. These control signals may include directions to spray the therapeutic at regular and irregular intervals of both long and short duration during the coating process. The control signals generated may depend upon the therapeutic being applied, the desired deposition of therapeutic on the implant, and the environmental conditions of the coating drum 31.

In addition to coming in direct contact with the rotatable drum, the implants may also be suspended above the surface of the drum 31 by compressed fluids (e.g., air and inert gas) being forced into the drum 31. These fluids may be forced into the drum through the perforations 32 and also through nozzles placed underneath the drum 31. The compressible fluids, which may be stored in the fluid source 38, may also be used for drying the implants after they have been coated. Moreover, in order to further facilitate the drying of the implants both the drum and the fluid may be heated through various available thermodynamic techniques.

The embodiment of FIG. 3 is also provided with a therapeutic recovery reservoir 34 for the recovery of therapeutic coating materials that fail to adhere to the medical implant(s) during the coating process. This recovery reservoir may generate a negative pressure to draw unused therapeutic out of the drum 31 and into the reservoir 34. This negative pressure may be continuously applied and may also be turned off and on during the coating process.

Also present in FIG. 3 is a storage media 36 and coating sources 371 and 372. The storage media may be used to provide information to the processor while the coating sources 371 and 372, which may be in fluid communication with the spray nozzle 39, may be used to supply coating material to the interior of the rotatable drum 31.

In addition to the non-volatile storage media described above, the processor 35 may also access the storage media 36 in order to receive instructions for operating and controlling the pan coater. This storage media 36 may contain instructions for performing each of the embodiments described herein as well as others that are also within the spirit and scope of the present invention. The storage media 36 may be one of numerous types of available storage media including both volatile (i.e. RAM) and non-volatile storage devices (i.e. ROM, CD ROM, EEPROM, Magnetic Media, etc.). Moreover, in addition to storing general instructions for operating the pan coater and coating the implants, the instructions may also be tailored to the specific implant being coated or the therapeutic being applied. For instance, the device may store information such as when implant A is being coated with therapeutic B, two applications of thirty seconds each may be required while if therapeutic C were used perhaps only a single forty-five second application would be necessary. The instructions may provide guidance on coating multiple implants and may also control the rotational speed of the drum 31, the pressure of the therapeutic being sprayed onto the implant, the various temperatures of the system and the fluids being employed, and the various sources of therapeutic being used. Moreover, pre-programmed instructions or other retained data may be unique to each medical implant and may account for the unique coating thickness required for each medical implant as well as for the number of medical implants present in the rotatable drum. Consequently, numerous types of information may be stored by the media.

Spray nozzle 39 may be in fluid communication with one or more coating sources. These coating sources may contain any one of several possible coatings to be placed on the medical implant. These coatings could include paclitaxel, a polymer with a suspended therapeutic, a non-thrombogenic agent, a lubricious material, a non-slippery material, a radiopaque agent, a radioactive agent, and a magnetic signature agent. These coatings could also include pharmaceutically active compounds, proteins, cells, oligonucleotides, ribozymes, anti-sense oligonucleotides, DNA compacting agents, gene/vector systems (i.e., any vehicle that allows for the uptake and expression of nucleic acids), nucleic acids (including, for example, recombinant nucleic acids; naked DNA, cDNA, RNA; genomic DNA, cDNA or RNA in a non-infectious vector or in a viral vector and which further may have attached peptide targeting sequences; antisense nucleic acid (RNA or DNA); and DNA chimeras which include gene sequences and encoding for ferry proteins such as membrane translocating sequences (“MTS”) and herpes simplex virus-1 (“VP22”)), and viral, liposomes and cationic and anionic polymers and neutral polymers that are selected from a number of types depending on the desired application. Non-limiting examples of virus vectors or vectors derived from viral sources include adenoviral vectors, herpes simplex vectors, papilloma vectors, adeno-associated vectors, retroviral vectors, and the like. Non-limiting examples of biologically active solutes include anti-thrombogenic agents such as heparin, heparin derivatives, urokinase, and PPACK (dextrophenylalanine proline arginine chloromethylketone); antioxidants such as probucol and retinoic acid; angiogenic and anti-angiogenic agents and factors; agents blocking smooth muscle cell proliferation such as rapamycin, angiopeptin, and monoclonal antibodies capable of blocking smooth muscle cell proliferation; anti-inflammatory agents such as dexamethasone, prednisolone, corticosterone, budesonide, estrogen, sulfasalazine, acetyl salicylic acid, and mesalamine; calcium entry blockers such as verapamil, diltiazem and nifedipine; antineoplastic/antiproliferative/anti-mitotic agents such as paclitaxel, 5-fluorouracil, methotrexate, doxorubicin, daunorubicin, cyclosporine, cisplatin, vinblastine, vincristine, epothilones, endostatin, angiostatin and thymidine kinase inhibitors; antimicrobials such as triclosan, cephalosporins, aminoglycosides, and nitorfurantoin; anesthetic agents such as lidocaine, bupivacaine, and ropivacaine; nitric oxide (NO) donors such as lisidomine, molsidomine, L-arginine, NO-protein adducts, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warafin sodium, Dicumarol, aspirin, prostaglandin inhibitors, platelet inhibitors and tick antiplatelet factors; vascular cell growth promotors such as growth factors, growth factor receptor antagonists, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; survival genes which protect against cell death, such as anti-apoptotic Bcl-2 family factors and Akt kinase; and combinations thereof. Cells can be of human origin (autologous or allogenic) or from an animal source (xenogenic), genetically engineered if desired. The delivery mediated is formulated as needed to maintain cell function and viability. Any modifications are routinely made by one skilled in the art.

Polynucleotide sequences useful in practice of the invention include DNA or RNA sequences having a therapeutic effect after being taken up by a cell. Examples of therapeutic polynucleotides include anti-sense DNA and RNA; DNA coding for an anti-sense RNA; or DNA coding for tRNA or rRNA to replace defective or deficient endogenous molecules. The polynucleotides of the invention can also code for therapeutic proteins or polypeptides. A polypeptide is understood to be any translation product of a polynucleotide regardless of size, and whether glycosylated or not. Therapeutic proteins and polypeptides include as a primary example, those proteins or polypeptides that can compensate for defective or deficient species in an animal, or those that act through toxic effects to limit or remove harmful cells from the body. In addition, the polypeptides or proteins that can be injected, or whose DNA can be incorporated, include without limitation, angiogenic factors and other molecules competent to induce angiogenesis, including acidic and basic fibroblast growth factors, vascular endothelial growth factor, hif-1, epidermal growth factor, transforming growth factor α and β, platelet-derived endothelial growth factor, platelet-derived growth factor, tumor necrosis factor α, hepatocyte growth factor and insulin like growth factor; growth factors; cell cycle inhibitors including CDK inhibitors; anti-restenosis agents, including p15, p16, p18, p19, p21, p27, p53, p57, Rb, nFkB and E2F decoys, thymidine kinase (“TK”) and combinations thereof and other agents useful for interfering with cell proliferation, including agents for treating malignancies; and combinations thereof. Still other useful factors, which can be provided as polypeptides or as DNA encoding these polypeptides, include monocyte chemoattractant protein (“MCP-1”), and the family of bone morphogenic proteins (“BMP's”). The known proteins include BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNA's encoding them.

The coatings that may be applied may also include: lubricious coatings to reduce the stress exerted on a stent during the stent's deployment; radiopaque coatings for identifying the location of the implants during and after implantation; radioactive agents that are useful in preventing tissue regrowth in and around implanted stents; and, magnetic coatings that can enable identification of the location of the implanted stent through Magnetic Resonance Imaging (MRI) techniques. The magnetic coatings may be created through the use of ferritic powders or paramagnetic powders such as Gadolinium or Disprosium. Moreover, in addition to having the coating material deposited in one coat or layer around the entire device, the pan coater may coat the medical implant with different layers of different thicknesses on the medical implant as may be required or desirable.

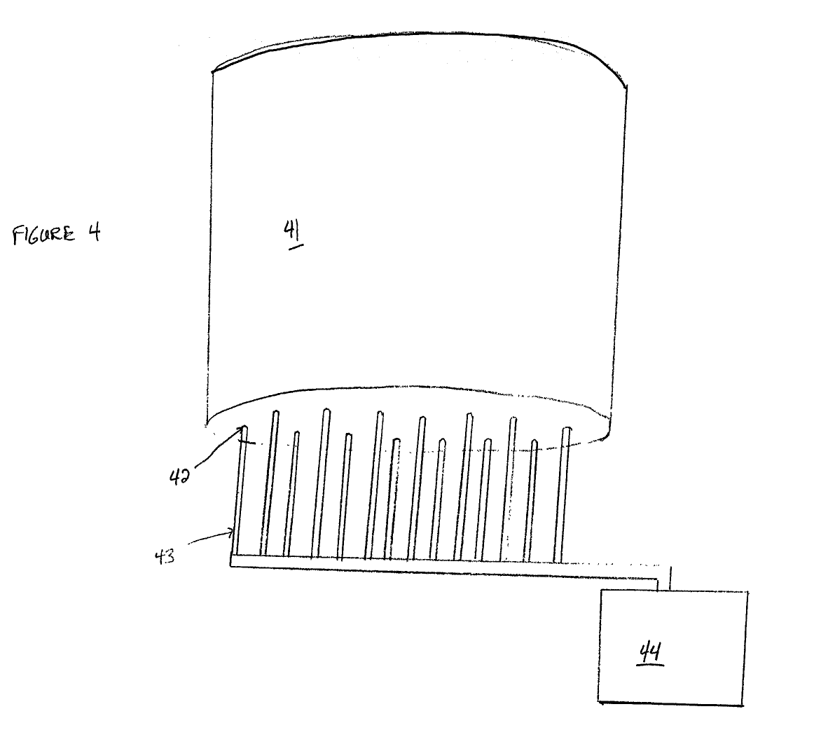

FIG. 4 shows a system for coating a medical implant using a pan coater equipped with an air suspension system for suspending the medical implant aloft in the pan coat drum 41 in accord with an alternative embodiment of the present invention. In this embodiment the pan coat drum 41, which may or may not be rotated, is used to coat a medical implant (not shown).

In this embodiment, the compressible fluid source 44 supplies high pressure, compressed fluid (i.e., air, inert gases, and other compressible fluids) to one or a group of fluid channels 43 that are in fluid communication with perforations 42 on the bottom side of the pan coat drum 41. Through these channels 43 and perforations 42 the compressible fluid should preferably create enough upward force in the drum 41 to suspend an implant being coated therein.

During use, a therapeutic coating may be introduced into the pan coat drum 41 by a spray nozzle (not shown) while the medical implants (not shown) are suspended by the upward flow of compressible fluid. The spray nozzle may be situated near the bottom of the drum 41 and may be used to introduce the therapeutic coating material into the upward flow of compressible fluid. Alternatively, there may be several spray nozzles situated on the perimeter of drum 41, each pointed and spraying inwardly to coat the medical implant. Regardless of the nozzle position, after the medical implants have been coated, the upward flow of compressible fluid may also assist in drying the therapeutic coating to the medical implants.

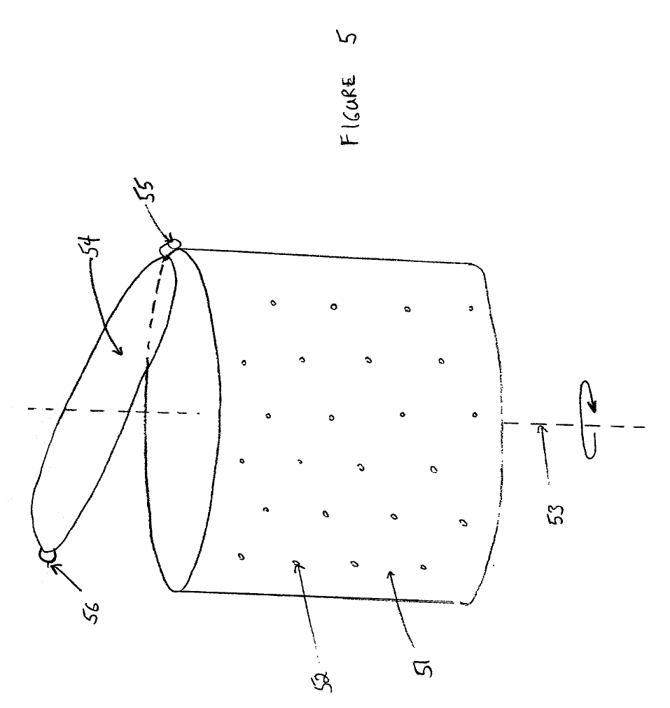

FIG. 5 shows an alternative embodiment of the present invention wherein a rotatable drum 51 is oriented about a vertical axis of rotation 53. The rotatable drum 51 in this embodiment has perforations 52 to allow compressible and incompressible fluid to flow in and out of it. The rotatable drum also has a closeable lid 54 with a handle 56, the lid attached by a hinge 55 to the top side of the rotatable drum 51. This lid 54 may be opened and closed during various times of the coating process to trap or otherwise retain therapeutic or compressible fluids in the drum.

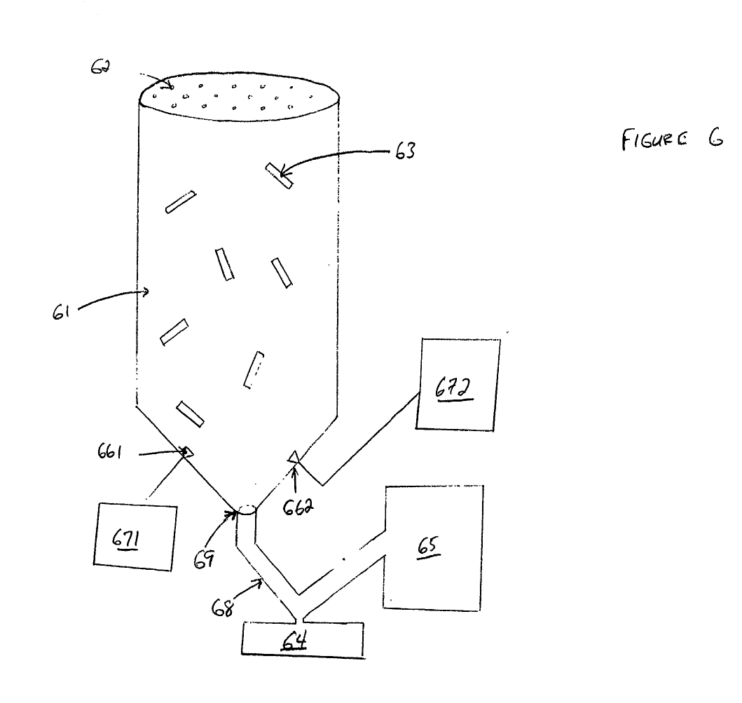

FIG. 6 shows another embodiment of the present invention. In this embodiment the pan coat drum 61, which may or may not be rotated during the coating process, is used to coat the medical implant(s) 63. The compressible fluid source 65 in this embodiment may be used to supply high pressure compressed fluid to a dual use channel 68 that is connected, via a passage 69, to the pan coat drum 61. As compressed fluid flows into the pan coat drum 61, via the passage 69, an upward flow of compressible fluid is created in the drum. As above, the upward flow of compressible fluid may be of sufficient strength to suspend, or hold aloft, the medical implant(s) 63 placed in the pan coat drum 61. Preferably after the medical implants 63 have been placed aloft, a therapeutic coating may be introduced into the pan coat drum 61 from coating source 671 by a spray nozzle 661 or, alternatively, coating source 672 by a spray nozzle 662 to coat the implants. Alternatively, the spraying of coating may begin before the implants are suspended within the drum 61.

The spray nozzles in the embodiment shown in FIG. 6 are situated near the bottom of the drum 61 so that the coating material is introduced into the upward flow of compressible fluid for delivery to the medical implant(s) 63. Alternatively, there may be several spray nozzles situated on the perimeter of drum 61 to coat the medical implant(s) 63. FIG. 6 also shows perforations 62 in the lid of drum 61 and a recovery reservoir 64. The recovery reservoir 64, which may be used for the collection of coating material that does not initially adhere to medical implant(s) 63, may be in fluid communication with drum 61 via dual use channel 68. The dual use channel 68 may be provided with a collection point to allow unused coating material to flow downward into recovery reservoir 64 without flowing into, or interfering with the compressible fluid source 65, which may also use dual use channel 68 sometime during the coating process.

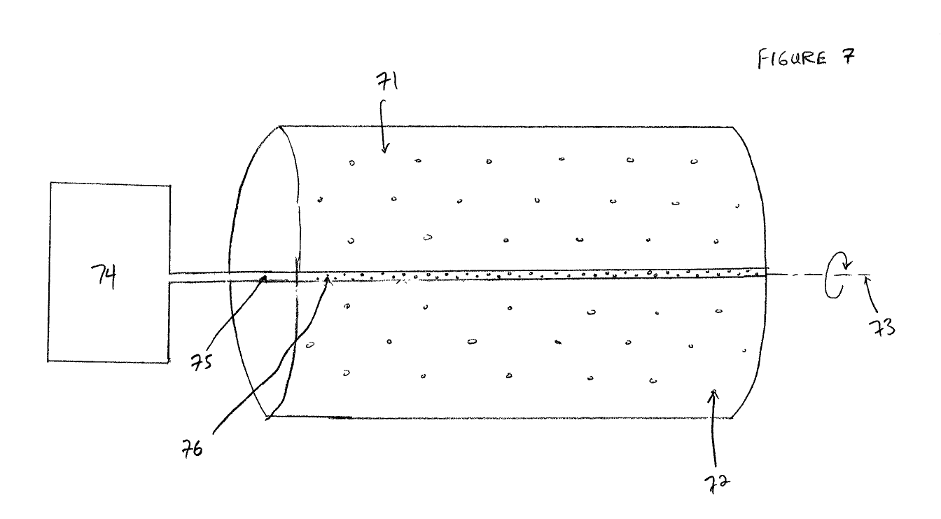

FIG. 7 shows an alternative embodiment of the present invention wherein a rotatable drum 71 is oriented about a horizontal axis of rotation 73. The rotatable drum 71 has perforations 72 to allow compressible and incompressible fluid to flow in and out of the rotatable drum 71. The rotatable drum 71 may also have a spray nozzle shaft 75 positioned on the axis of rotation 73 of the drum 71. Spray nozzle shaft 75 has spray jets 76 and is in fluid communication with coating source 74. Therefore, in use, therapeutic may be forced down the shaft 75 and out the jets 76 to coat the medical implants located within the drum 71.

A method for using a pan coater for coating a medical implant is provided herein. While several embodiments have been discussed, others, within the invention's spirit and scope, are also plausible. For example, while using a pan coater to apply a single coat to a medical implant is described, it may be advantageous to apply multiple coats of either the same or different materials, simultaneously or consecutively, to the medical implant. Alternatively, while one pan coater is described in each of the above embodiments more than one pan coater may also be employed. In this alternative embodiment, the multiple pan coaters may work consecutively to apply different coatings during different process steps. Moreover, the pan coater in any of these embodiments may be used for other indiscriminate coating applications, including cleaning the medical implant, applying a coating to a medical implant that has been selectively masked (wherein the mask may or may not be removed at a later time), and applying a material that reacts with a second material to etch the medical implant (wherein the second material has been selectively applied beforehand to specific areas to be etched of the medical implant).

What is claimed is:

1. A method of coating a medical implant comprising:
placing a medical implant into a rotatable drum;
tumbling the medical implant in the drum for a predetermined amount of time; and
interfacing a therapeutic with the tumbling medical implant.

2. The method of claim 1, further comprising:
drying the therapeutic on the medical implant.

3. The method of claim 2, wherein drying the therapeutic on the medical implant includes spraying an inert gas into the drum.

4. The method of claim 1, further comprising:
suspending the medical implants above an internal surface of the drum.

5. A method for applying a coating to a medical implant comprising:
providing a pan coater, the pan coater including a drum having at least a first opening;
placing a medical implant in the drum of the pan coater;
rotating the drum to tumble the medical implant;
spraying a therapeutic into the drum to coat the medical implant; and
removing the medical implant from the drum.

6. The method of claim 5, wherein the drum is a drum rotatable about its longitudinal axis.

7. The method of claim 5, further comprising:
forcing a compressible fluid from a compressible fluid source into the drum;
circulating the compressible fluid in the drum; and
waiting until the therapeutic on the medical implant is dry before removing the medical implant from the drum.

8. The method of claim 7, wherein spraying the therapeutic into the drum is repeated at least once.

9. The method of claim 7, further comprising:
heating the compressible fluid in the compressible fluid source prior to forcing the compressible fluid into the drum.

10. The method of claim 9, wherein the compressible fluid in the compressible fluid source is heated to a temperature in the range of 20 to 70 degrees centigrade.

11. The method of claim 9, wherein the compressible fluid in the compressible fluid source is heated to a temperature associated with a working temperature of the therapeutic.

12. The method of claim 5, further comprising:
drawing a compressible fluid into the drum.

13. The method of claim 5, further comprising:
heating the rotatable drum after spraying the therapeutic into the drum.

14. The method of claim 5, wherein the pan coater is provided with a compressible fluid suspension system that forces a compressible fluid into the drum with a force sufficient to maintain the medical implant aloft in the drum.

15. The method of claim 14, wherein the compressible fluid suspension system uses an inert gas to maintain the medical implants aloft.

16. The method of claim 14, further comprising:
periodically activating the compressible fluid suspension system.

17. The method of claim 5, wherein the drum has perforations on an outer surface.

18. The method of claim 17, further comprising:
passing therapeutic through the perforations; and
passing compressible fluid through the perforations.

19. The method of claim 5, further comprising:
recycling therapeutic that did not adhere to the implant during spraying.

20. A computer readable medium storing instructions for operating a pan coater for coating a medical implant, the instructions comprising directions for the pan coater to:
rotate a drum to tumble a medical implant;
spray a first therapeutic into the drum through a spray nozzle while rotating the drum; and
stop the drum from rotating.

21. The computer readable medium of claim 20, storing further directions for the pan coater to:
force a compressible fluid into the drum after spraying the first therapeutic into the drum.

22. The computer readable medium of claim 21, storing further directions for the pan coater to:
heat the compressible fluid prior to forcing the compressible fluid into the drum.

23. The computer readable medium of claim 20 storing further directions for the pan coater to:
draw a compressible fluid out of the drum through a compressible fluid exhaust opening.

24. The computer readable medium of claim 20 storing further directions for the pan coater to:
spray a second therapeutic into the drum after a medical implant has been placed into the drum.

25. A method for applying a coating to a medical implant comprising:
providing a pan coater, the pan coater including a drum having at least a first opening;
placing a medical implant in the drum of the pan coater;
injecting a compressible fluid into the drum with a force sufficient to maintain the medical implant aloft in the drum to tumble the medical implant;
spraying a therapeutic into the drum to coat the medical implant; and
removing the medical implant from the drum.

26. The method of claim 25, wherein the compressible fluid is an inert gas.

27. The method of claim 25, wherein the compressible fluid is also for drying the therapeutic on the medical implant.

28. The method of claim 25, further comprising:
periodically injecting the compressible fluid into the drum.
